(19) United States
(12) Patent Application Publication
(10) Pub. No.: US 2025/0058067 A1
(43) Pub. Date: Feb. 20, 2025

(54) RESUSCITATION DEVICE AND METHOD OF USING

(71) Applicant: Ethan R. Zahalsky, Parkland, FL (US)

(72) Inventor: Ethan R. Zahalsky, Parkland, FL (US)

(21) Appl. No.: 18/365,411

(22) Filed: Aug. 17, 2023

(51) Int. Cl. A61M 16/00; A61M 16/06

(52) U.S. Cl. CPC A61M 16/0066; A61M 16/0078; A61M 16/0605

(57) ABSTRACT

An improved air resuscitation device to provide air to a patient with impaired breathing has a fan assembly and a flexible mouth and nasal opening cover. The fan assembly includes a fan and a motor, one or more batteries to power the fan, a switch to connect to the fan to turn the fan on or off, a controller with a circuit board having a timer and flow controller. The device, when attached to the patient and switched on, powers the fan to deliver air into and through the mouth and nasal opening cover into the patient's airway for a first set period of time and the fan pauses allowing the air to vent out of the fan assembly for a second set period of time and then the fan restarts and pauses repeatedly automatically providing air.

### TECHNICAL FIELD

The present invention provides an improved device to provide air to a person requiring breathing assistance, more particularly to a device that has a fan to introduce air into the patient and provide needed oxygen for a person unable to breathe without assistance.

### BACKGROUND OF THE INVENTION

When people are in breathing distress and are unable to breathe on their own, they will quickly die from a lack of oxygen. In some cases, the person may be in cardiac arrest requiring Cardio-Pulmonary Resuscitation (CPR). The chest compressions work to get the heart pumping and mouth to mouth resuscitation can be provided intermittently between the chest compressions to get air into the lungs.

For safety reasons, the airway resuscitation is commonly done by a device shown in FIGS. 1, 2A and 2B that fits over the mouth of the patient and has a bag that has air that can be squeezed to push air into the patient's lungs. The bag automatically refills with air to allow repeated inflows of air to the lungs. This commonly used device avoids the person assisting from contacting the patient's mouth and reduces the risk of contracting any disease carried by the patient. As shown, the device can be connected to an oxygen reservoir bag.

This rather simple bag device for resuscitation is commonly used. The device requires a level of expertise to know when and how often to squeeze the air-filled bag. Ideally, a resuscitation device for such purposes would be able to automatically provide repeated air inflow volume without requiring the use to be skilled in emergency breathing assistance.

The present invention provides such a device which can not only provide inflows of air, but in one embodiment includes inflow of fresh air and extraction of used air in an automated or timed pattern as described hereinafter.

### SUMMARY OF THE INVENTION

An improved air resuscitation device to provide air to a patient with impaired breathing has a fan assembly and a flexible mouth and nasal opening cover. The flexible mouth and nasal opening cover is configured to encircle a mouth and nasal opening of the patient. The fan assembly is configured to connect to the flexible mouth and nasal opening covering. The fan assembly has a first connection portion attached to the mouth and nasal opening cover and a second portion having an air vent opening for receiving air into and exhausting air out of the fan assembly. The fan assembly includes a fan and a motor, one or more batteries to power the fan, a switch to connect to the fan to turn the fan on or off, a controller with a circuit board having a timer and flow controller. The device, when attached to the patient and switched on, powers the fan to deliver air into and through the mouth and nasal opening cover into the patient's airway for a first set period of time and the fan pauses allowing the air to vent out of the fan assembly for a second set period of time and then the fan restarts and pauses repeatedly automatically providing air.

The improved air resuscitation device further has an air filter covering the air vent opening. The controller circuit board is configured to reverse the fan rotation during the pause causing the fan to exhaust air from the patient prior to restarting the fan rotation in the air inflow direction to send air into the patient.

In one embodiment, the fan assembly has a port for connecting an oxygen chamber to provide oxygen to the patient.

The fan delivers air inflow at a pressure of 30 to 40 cm of H2O. The air inflow during the first set period of time is between 5 and 10 seconds and the second set period of time has a pause time for venting of 2 to 5 seconds. The device controller can be set for a patient type including and adult male, adult female, an adolescent child, or an infant varying the pressure, time and air volume according to the patient type. For an adult male, the air inflow is at least 500 cm³ of air per minute. The device is set for 10 to 12 respirations per minute for a child or infant. Alternatively, the device can be designed with variable settings that can be adjusted at the user's discretion. The device parameters could be altered, decreasing or increasing the range, frequency, volume, etc. at the manufacturer's discretion.

Preferably, the fan assembly is detachable from the mouth and nasal opening cover and replaceable with an optional flexible air bag.

In another embodiment of the invention, an improved air resuscitation adapter device to provide air to a patient with impaired breathing is disclosed. The adapter device is a fan assembly. The fan assembly can be connected or otherwise configured to be attached to a mouth and nasal opening cover at a first end of the fan assembly. The fan assembly is configured to pass air into and through the mouth and nasal opening cover. The fan assembly includes a fan motor, one or more batteries to power the fan, a switch to connect to the fan to turn the fan on or off, a controller with a circuit board having a timer and flow controller. The adapter device, when attached to the mouth and nasal opening cover, positioned over the patient's mouth and nasal opening, and switched on, powers the fan to deliver air through the mouth and nasal opening cover into the patient's airway for a first set period of time and the fan pauses allowing the air to vent for a second set period of time and then the fan restarts and pauses repeatedly automatically providing air.

The adapter device is detachable from the mouth and nasal opening cover and replaceable with an optional flexible air bag.

The present invention allows for an automated method to provide air to a patient having breathing distress.

A method of providing air to a patient with impaired breathing, the method comprising the steps of: clearing the patient's airway; identifying the type of patient; setting the device to the type of patient; providing the resuscitation device; attaching the device to the patient and turning the switch on; monitoring the patient and when the patient regains normal breathing, shutting off and removing the device.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

A bag valve mask, sometimes known by the proprietary name Ambu bag or generically as a manual resuscitator or “self-inflating bag”, is a hand-held device commonly used to provide positive pressure ventilation to patients who are not breathing or not breathing adequately.

Cardio-Pulmonary Resuscitation is a procedure designed to restore normal breathing after cardiac arrest that includes the clearance of air passages to the lungs, mouth-to-mouth method of artificial respiration, and heart massage by the exertion of pressure on the chest.

### BRIEF DESCRIPTION OF THE DRAWINGS

The invention will be described by way of example and with reference to the accompanying drawings in which:

FIG. 1 is a prior art bag valve mask being used.

FIG. 2A is a plan view of a prior art bag valve mask with an optional oxygen bag.

FIG. 2B is a view of the bag valve mask of FIG. 2A in use.

FIG. 3 is a plan view of the device made according to the present invention.

FIG. 4 is an exploded perspective view of the present invention.

### DETAILED DESCRIPTION OF THE INVENTION

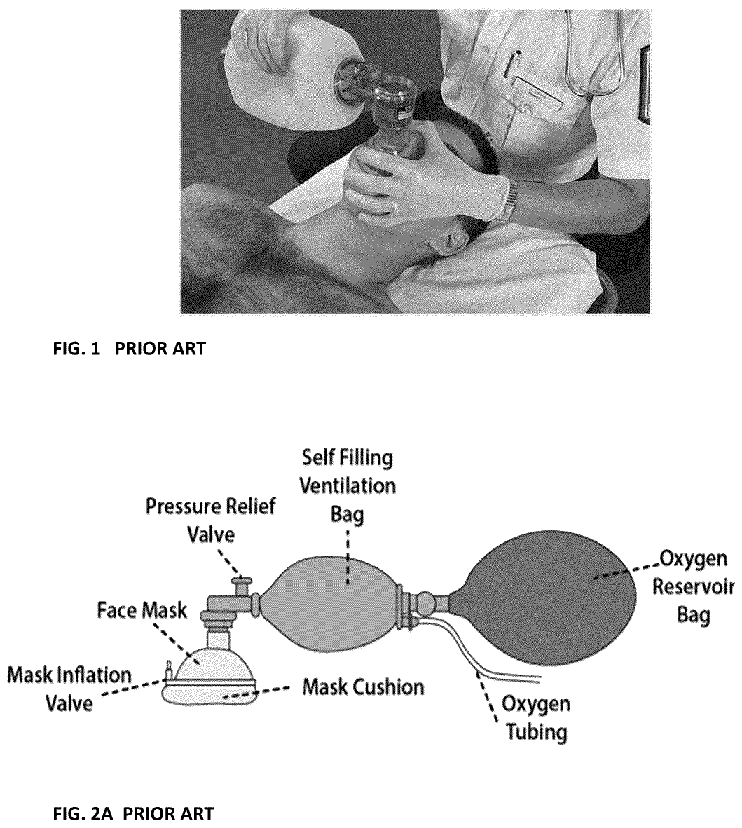

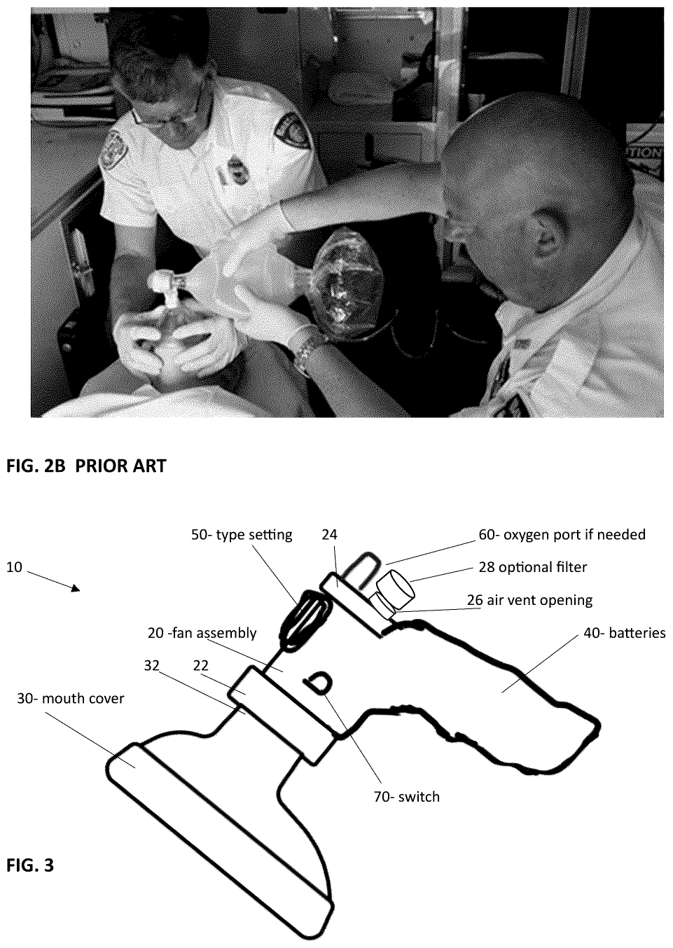

The present invention is an improvement over the prior art devices shown in FIGS. 1, 2A and 2B in that the flexible and squeezable bag for providing air to a patient in breathing distress has been replaced by a fan assembly.

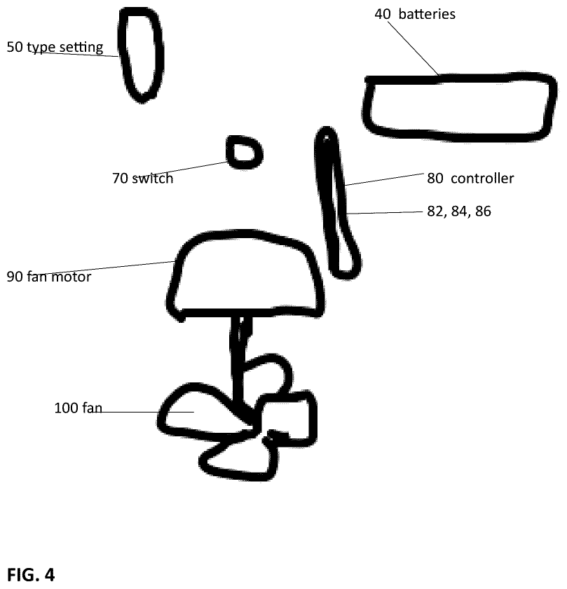

With reference to FIGS. 3 and 4, the fan assembly device 10 is shown wherein the flexible bag has been replaced by the fan assembly 20. The air resuscitation device 10 provides air to the patient with impaired breathing. The device 10 has a fan assembly 20 and a flexible mouth and nasal opening cover 30. The flexible mouth and nasal opening cover 30 is configured to encircle a mouth and nasal opening of the patient with the impaired breathing. When this cover 30 is placed over and encircling the mouth and nasal area, the mouth and nasal opening cover 30 provides a pathway such that air can be delivered to the patient to assist in the patient's breathing. As shown, the fan assembly 20 is configured to be connected to a port on the flexible mouth and nasal opening cover 30. The fan assembly 20 has a first connection portion 22 that attaches to the port 32 of the flexible mouth and nasal opening cover 30 and can be attached and detached as desired. Once attached, the fan assembly 20 and flexible mouth and nasal opening cover 30 provide the necessary components for resuscitation. The fan assembly 20 connects to the flexible mouth and nasal opening cover 30 at a first connection portion 22, a second portion 24 is provided having an air vent opening 26 for receiving air into and exhausting air out of the fan assembly 20. The fan assembly 20 has a fan 100, a fan motor 90, one or more batteries 40 to power the fan 100, a switch 70 to connect to the fan to turn the fan on or off and a controller 80 with a circuit board 82 having a timer 84 and flow control 86 as shown in FIG. 3 and the exploded view of FIG. 4.

Accordingly, when the device 10 is attached to the patient and switched on, it powers the fan 100 to deliver air into and through the flexible mouth and nasal opening cover 30 into the patient's airway for a first set period of time. The fan 100 pauses allowing air to vent for a second period of time and then the fan restarts and pauses repeatedly automatically providing air.

In one embodiment, the device 10 has an air filter 28 covering the vent opening 26 the fan assembly 20. This filter 28 ensures that contaminants are not sucked into the fan 100 and delivered into the patient's lungs. The filter 28 is sufficiently open however so the air can easily vent outward through the air filter 28 when attached to the fan assembly 20.

A controller circuit board 82 is provided that is configured to reverse the fan rotation during the pause causing the fan to exhaust air from the patient prior to restarting the fan rotation in the air inflow direction to send air into the patient. This ability to suction air from the patient is extremely important and an improvement over the prior art system. In normal breathing air is sucked in providing oxygen to the lungs and then expelled by exhausting that air out. In such circumstances, the chest and diaphragm area and lungs expand greatly during air intake and then contract allowing the air to be pushed out of the patient. This normal breathing practice is interrupted when a patient has breathing distress and is unable to breathe effectively on their own. Under such circumstances, the air coming into the patient is difficult to discharge or exhaust. Accordingly, the air becomes stagnant with a lack of oxygen that can render the patient with breathing distress in a serious situation where a lack of oxygen can affect all other bodily functions.

The present invention provides a fan that provides a strong enough air flow volume and pressure to provide 30 to 40 cm of water pressure to the patient during the air inflow period. This air inflow period allows the lungs to fill sufficiently to provide oxygen to the patient, however, when the fan is off during the venting pause period, adequate air removal is not always guaranteed. Therefore, it is ideal that the controller circuit 82 provided with the fan assembly be configured such that during the pause time, the fan motor 90 can be reversed causing the fan blades to reverse direction and exhaust air out of the patient during this pause period. This pausing allows for some of the air to be literally sucked out of the patient during the pause period and then upon starting air inflow a larger volume of fresh air can be provided to the patient to provide more oxygen to the patient than he or she would otherwise receive during a normal bagging procedure.

It is noted that the controller circuit 82 is designed in such a fashion that the controller 80 can be set for a specific patient type. The setting could be 1-4 in increments for infant up to adult male. For example, an adult male, an adult female, an adolescent child or an infant. The selection allows the fan to vary the pressure time and air volumes according to the patient type. For example, and adult male will have an air inflow of at least 500 cm³ of air per minute to adequately resuscitate the patient. These resuscitations occur 5 or 6 times per minute and this volume of air which is typically 500-600 cm³ per minute is sufficient to provide adequate oxygen to the patient assisting his breathing. The device also can be set for 10 to 12 respirations per minute for a child or infant at a much lower volume. This allows smaller breaths to be taken on a child or infant. As previously mentioned, the parameters disclosed are simply a recommendation, alternatively other parameters could be employed.

As one size does not fit all in air resuscitation, the prior art air bag systems rely on the operator to evaluate the patient based on patient type and provide the adequate squeezing of the bag so the patient receives the correct amount of air. This requires some expertise. The fan assembly of the present invention does this automatically by selecting the patient type and turning the switch on. The patient will the receive the preferred amount of air volume at the preferred amount of pressure. This is a great improvement because a person not trained in providing air resuscitation can easily use the device by simply selecting the proper settings for the device prior to initiating use.

As shown, the device 10 when attached to the flexible mouth and nasal opening cover 30 creates the entire device. However, it is noted that in one embodiment, the invention is simply a resuscitation adapter device that is designed to be coupled to a flexible mouth and nasal opening cover. This adapter device is the fan assembly 20 itself. In this case the fan assembly 20 can be attached to any mouth and nasal cover opening through a port at a first connection end 22 of the fan assembly 20. The fan assembly 20 has all the components as previously described. This adapter device is detachable from the flexible mouth and nasal opening cover and can be replaced by an optional flexible air bag to provide manual squeezing if so desired as illustrated in the prior art figures.

Ideally, the present invention fan assembly has a battery capacity that will provide air breathing for an extended period of time, preferably over an hour if needed. However, this should allow the patient to resume normal breathing within much shorter periods of time. Should the battery be depleted or the fan assembly malfunction, the device can be used as a normal breathing bag by manually squeezing or pumping the bag. The fan device can also be a device that attaches to a normal breathing apparatus to assist with the breathing. It does not have to be a stand alone set, but can attach to an already existing one.

As described, the present invention, either in the first embodiment entire assembly or the adapter embodiment which is just the fan assembly, provides a method of providing air to a patient with impaired breathing which includes the method steps of clearing the patient's airway, identifying the type of patient, providing the resuscitation device, setting the device to the type of patient, attaching the device to the patient and turning the switch on, and monitoring the patient and when the patient regains normal breathing, shutting off and removing the device. After use, the mouth and nasal opening cover is discarded if it is disposable, the fan assembly is cleaned with a disinfectant and a new filter is provided making it safe to be reused.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. An improved air resuscitation device to provide air to a patient with impaired breathing, the device comprising:
a flexible mouth and nasal opening cover configured to encircle a mouth and nasal opening of the patient;
a fan assembly connected to the flexible mouth and nasal opening covering, the fan assembly having a first connection portion attached to the mouth and nasal opening cover and a second portion having an air vent opening for receiving air into and exhausting air out of the fan assembly, the fan assembly including a fan and a motor, one or more batteries to power the fan, a switch to connect to the fan to turn the fan on or off, a controller with a circuit board having a timer and flow controller; and
wherein the device, when attached to the patient and switched on, powers the fan to deliver air into and through the mouth and nasal opening cover into the patient's airway for a first set period of time and the fan pauses allowing the air to vent out of the fan assembly for a second set period of time and then the fan restarts and pauses repeatedly automatically providing air.

2. The improved air resuscitation device of claim 1, further comprises:
an air filter covering the air vent opening.

3. The improved air resuscitation device of claim 1, wherein the controller circuit board is configured to reverse the fan rotation during the pause causing the fan to exhaust air from the patient prior to restarting the fan rotation in the air inflow direction to send air into the patient.

4. The improved air resuscitation device of claim 1, wherein the fan assembly has a port for connecting an oxygen chamber to provide oxygen to the patient.

5. The improved air resuscitation device of claim 1, wherein the fan delivers air inflow at a pressure of 30 to 40 cm of H₂O.

6. The improved air resuscitation device of claim 1, wherein the air inflow during the first set period of time is between 5 and 10 seconds and the second set period of time being a pause time for venting of 2 to 5 seconds.

7. The improved air resuscitation device of claim 1, wherein the device controller can be set for a patient type including and adult male, adult female, an adolescent child, or an infant varying the pressure, time and air volume according to the patient type.

8. The improved air resuscitation device of claim 7, wherein for an adult male, the air inflow is at least 500 cm³ of air per minute.

9. The improved air resuscitation device of claim 7, wherein the device is set for 10 to 12 respirations per minute for a child or infant.

10. The improved air resuscitation device of claim 1, wherein the fan assembly is detachable from the mouth and nasal opening cover and replaceable with an optional flexible air bag.

11. An improved air resuscitation adapter device to provide air to a patient with impaired breathing, the adapter device comprising:
a fan assembly connected or otherwise configured to be attached to a mouth and nasal opening cover at a first end of the fan assembly, the fan assembly being configured to pass air into and through the mouth and nasal opening cover, the fan assembly including, a fan motor, one or more batteries to power the fan, a switch to connect to the fan to turn the fan on or off, a controller with a circuit board having a timer and flow controller; and
wherein the adapter device, when attached to the mouth and nasal opening cover, positioned over the patient's mouth and nasal opening, and switched on, powers the fan to deliver air through the mouth and nasal opening cover into the patient's airway for a first set period of time and the fan pauses allowing the air to vent for a second set period of time and then the fan restarts and pauses repeatedly automatically providing air.

12. The improved air resuscitation adapter device of claim 11, wherein the adapter device is detachable from the mouth and nasal opening cover and replaceable with an optional flexible air bag.

13. A method of providing air to a patient with impaired breathing, the method comprising the steps of:
clearing the patient's airway;
identifying the type of patient;
providing the device of claim 1;
setting the device to the type of patient;
attaching the device to the patient and turning the switch on;
monitoring the patient and when the patient regains normal breathing, shutting off and removing the device.
